(19) United States
(12) Patent Application Publication
(10) Pub. No.: US 2008/0125658 A1
(43) Pub. Date: May 29, 2008

(54) Low-profile acoustic transducer assembly

(75) Inventors: Warren Lee, Niskayuna, NY (US); Douglass Glenn Wildes, Ballston Lake, NY (US); David Martin Mills, Niskayuna, NY (US)

Correspondence Address: GENERAL ELECTRIC COMPANY (PCPI);C/O FLETCHER YODER, P. O. BOX 692289, HOUSTON, TX, 77269-2289, US

(73) Assignee: General Electric Company

(21) Appl. No.: 11/515,073

(22) Filed: Sep. 1, 2006

(51) Int. Cl. A61B 8/12; B06B 1/06; H04R 31/00

(52) U.S. Cl. 600/459

(57) ABSTRACT

A transducer assembly is presented. The transducer assembly includes an acoustic layer having a first side and a second side, opposite the first side. Furthermore, the transducer assembly includes at least one matching layer disposed on the first side of the acoustic layer. Additionally, the transducer assembly includes a dematching layer disposed on the second side of the acoustic layer, where the dematching layer has an acoustic impedance greater than an acoustic impedance of the acoustic layer, and where the transducer assembly does not include a backing layer that is highly attenuative.

### BACKGROUND

The invention relates generally to acoustic transducers, and more specifically to a transducer assembly for use in a probe configured for imaging in space-constrained applications.

Acoustic transducers have found application in medical imaging where an acoustic probe is held against a patient and the probe transmits and receives ultrasound waves. The received energy may, in turn, facilitate the imaging of the internal tissues of the patient. For example, transducers may be employed to image the heart of the patient.

Catheter-based ultrasonic imaging techniques are interventional procedures that generally involve inserting a probe, such as an imaging catheter, into a vein, such as the femoral vein or an artery. As will be appreciated, catheter-based ultrasonic imaging techniques may be employed for monitoring and/or directing treatment of atrial fibrillation, for example, where atrial fibrillation is one of the most common cardiac arrhythmias encountered in clinical practice. Consequently, it is highly desirable that transducer assemblies used in catheter-based imaging catheters are capable of two-dimensional and/or real-time three-dimensional imaging. Such applications are quite demanding, requiring very small transducer packages that can nevertheless collect large amounts of information.

A typical ultrasound probe includes a miniaturized transducer assembly disposed at a distal tip of the probe. The probe may include, for example, a one-dimensional phased array transducer. Furthermore, the transducer assembly is designed such that a plurality of transducer elements is disposed along a longitudinal and/or transverse axis of the probe. However, the elevational dimension of each of the plurality of transducer elements is constrained by the diameter of the probe. As will be appreciated, for a one-dimensional transducer array with elements arranged along the longitudinal axis of the probe, the elevation resolution is dependent upon the aperture size or physical extent of the transducer element in the elevational dimension. The larger the elevational size of the element, the better the resolution. For a one-dimensional array transducer producing a two-dimensional image, the elevational resolution affects the image contrast. The probe environment imposes a severe size constraint in the elevation dimension. Thus, designs which allow the elevational dimension of the element to be maximized would result in improved image quality.

Previously conceived solutions to this problem have incorporated transducer assemblies developed for use in non-invasive probes. These conventional transducer assemblies typically include a backing layer designed to absorb the acoustic energy propagating towards the rear of the transducer element and/or to provide mechanical support for the transducer assembly. Unfortunately, because such backing layers are relatively thick, the thickness of the transducer assembly is considerably increased. Consequently, the elevational aperture of the probe is disadvantageously decreased. In addition, the probe may also include multi-wire cabling configured to couple the transducer assembly to the rest of an imaging system. However, the high density of interconnections required to address each transducer element in a transducer array and the thickness of the transducer package disadvantageously result in poor space efficiency of the transducer assemblies. Additionally, the imaging resolution and sensitivity of these probes have suffered due to the presence of such transducer assemblies.

There is therefore a need for a design of a transducer assembly capable of two-dimensional imaging and/or real-time three-dimensional imaging for use in a probe employed in space-constrained applications such as intracardiac imaging. In particular, there is a significant need for a design of a low-profile transducer assembly that maximizes elevational aperture size, thereby resulting in enhanced image resolution and sensitivity of the probe. Also, it would be desirable to develop a simple and cost-effective method of fabricating a transducer assembly capable of real-time three-dimensional imaging.

### BRIEF DESCRIPTION

Briefly, in accordance with aspects of the invention, a transducer assembly is presented. The transducer assembly includes an acoustic layer having a first side and a second side, opposite the first side. Further, the transducer assembly also includes at least one matching layer disposed on the first side of the acoustic layer. In addition, the transducer assembly includes a dematching layer disposed on the second side of the acoustic layer. The dematching layer has an acoustic impedance greater than an acoustic impedance of the acoustic layer. Further, the transducer assembly does not include a backing layer that is highly attenuative, thereby reducing the overall thickness of the assembly.

In accordance with further aspects of the invention, a transducer assembly configured for use in an invasive probe is presented. The transducer assembly consists of an acoustic layer having a first side and a second side opposite the first side, at least one matching layer disposed on the first side of the acoustic layer, and a dematching layer disposed on the second side of the acoustic layer. The dematching layer has an acoustic impedance greater than an acoustic impedance of the acoustic layer. A flexible interconnect layer is also provided that comprises at least one conductive element disposed on a substrate. The conductive element is configured to facilitate coupling the transducer elements to a cable assembly or electronics.

In accordance with yet another aspect of the invention, an invasive probe configured to image an anatomical region is provided that includes an outer envelope sized and configured to be removably inserted into a patient. The invasive probe includes a transducer assembly disposed in the outer envelope. The transducer assembly includes an acoustic layer having a first side and a second side, opposite the first side, at least one matching layer disposed on the first side, and a dematching layer disposed on the second side. The dematching layer has an acoustic impedance greater than an acoustic impedance of the acoustic layer. The transducer assembly does not include a backing layer that is highly attenuative.

In accordance with further aspects of the invention, a system is provided that includes an acquisition subsystem configured to acquire image data. The acquisition subsystem comprises an invasive probe configured to image an anatomical region. The invasive probe is configured as summarized above. Additionally, the system includes a processing subsystem in operative association with the acquisition subsystem and configured to process the image data acquired via the acquisition subsystem.

In accordance with further aspects of the invention, a method for forming a transducer assembly is presented. The method includes forming a stacked structure that includes an acoustic layer having a first side and a second side opposite the first side, at least one matching layer disposed on the first side of the acoustic layer, and a dematching layer disposed on the second side of the acoustic layer, where the dematching layer has an acoustic impedance greater than an acoustic impedance of the acoustic layer, where the transducer assembly does not include a backing layer that is highly attenuative. In addition, the method includes bonding the stacked structure to an interconnect layer, or a substrate, or both. Furthermore, the method includes dicing the stacked structure to form a plurality of transducer elements.

### DRAWINGS

These and other features, aspects, and advantages of the invention will become better understood when the following detailed description is read with reference to the accompanying drawings in which like characters represent like parts throughout the drawings, wherein:

FIG. 1 is a block diagram of an exemplary ultrasound imaging system, in accordance with aspects of the present technique;

FIG. 2 illustrates a portion of an invasive probe including an exemplary transducer assembly for use in the system illustrated in FIG. 1, in accordance with aspects of the present technique;

FIG. 3 is a diagrammatic illustration of the ultrasound imaging system illustrated in FIG. 1;

FIG. 4 is a perspective view of an exemplary embodiment of a low-profile transducer assembly for use in the system illustrated in FIG. 1, in accordance with aspects of the present technique;

FIG. 5 illustrates an exemplary embodiment of an invasive probe including the low-profile transducer assembly illustrated in FIG. 4, in accordance with aspects of the present technique;

FIG. 6 is an illustration of an exemplary invasive probe including the low-profile transducer assembly illustrated in FIG. 4, in accordance with aspects of the present technique;

FIG. 7 is an end view of the invasive probe illustrated in FIG. 6, in accordance with aspects of the present technique;

FIG. 8 is an end view of an invasive probe depicting a mode of interconnection, in accordance with aspects of the present technique;

FIG. 9 is an end view of an invasive probe depicting an alternate mode of interconnection, in accordance with aspects of the present technique;

FIG. 10 is an end view of the invasive probe illustrated in FIG. 6 depicting additional components, in accordance with aspects of the present technique;

FIG. 11 is a graphical representation of exemplary simulation results depicting the effect of various materials disposed to the rear of a dematching layer in the low-profile transducer assembly illustrated in FIG. 4, in accordance with aspects of the present technique;

FIG. 12 is a series of schematic sectional views of progressive formation of a low-profile transducer assembly in an exemplary method in accordance with aspects of the present technique;

FIG. 13 is a similar series of schematic sectional views of another exemplary method for forming a low-profile transducer assembly, in accordance with aspects of the present technique; and

FIG. 14 is a further series of schematic sectional views of progressive formation of a low-profile transducer assembly in accordance with yet another exemplary method in accordance with aspects of the present technique.

### DETAILED DESCRIPTION

As will be described in detail hereinafter, a transducer assembly capable of real-time two-dimensional or three-dimensional imaging sized and configured for use in an invasive probe employed in space-constrained applications, such as intracardiac imaging, and methods of forming such an array are presented. By employing the invasive probe having the exemplary transducer assembly, a relatively high-quality two-dimensional or three-dimensional image with improved contrast resolution may be obtained. Based on the image data acquired by the invasive probe, a user may assess need for therapy in an anatomical region and direct the therapy via the invasive probe. In accordance with aspects of the present technique, it may be noted that the invasive probe may be used for imaging a region of interest and directing therapy. Alternatively, a first invasive probe may be used for imaging the region of interest, while at least a second probe may be configured to direct therapy to the region of interest.

Although, the exemplary embodiments illustrated hereinafter are described in the context of a medical imaging system, it will be appreciated that use of the probe with improved image quality and contrast resolution in industrial applications is also contemplated in conjunction with the present technique. For example, the exemplary embodiments illustrated and described hereinafter may find application in industrial borescopes that are employed for thickness monitoring, interface monitoring, or crack detection.

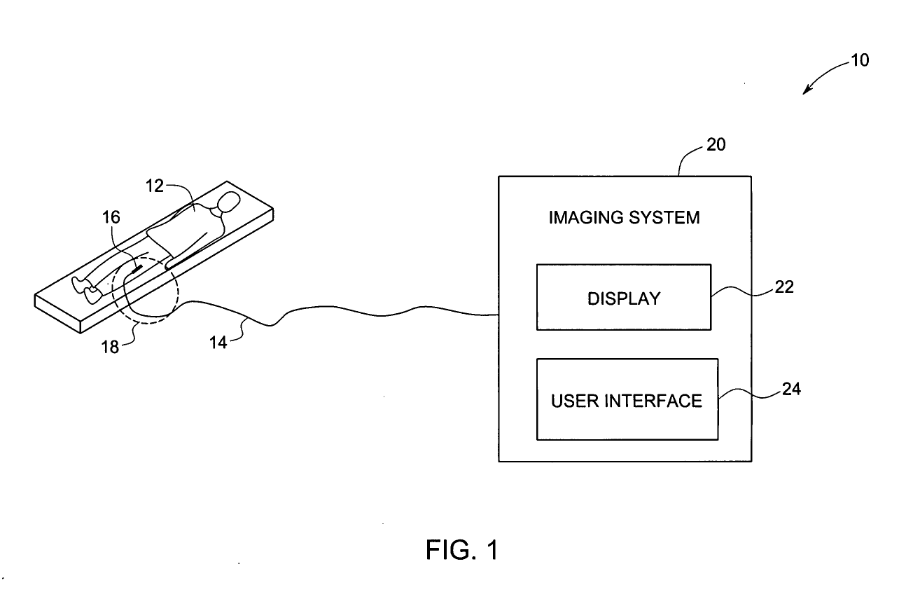

FIG. 1 is a block diagram of an exemplary system 10 for use in imaging, in accordance with aspects of the present technique. As will be appreciated by those skilled in the art, the figures are for illustrative purposes and are not drawn to scale. The system 10 may be configured to facilitate acquisition of image data from a patient 12 via a probe 14. In other words, the probe 14 may be configured to acquire image data representative of a region of interest in the patient 12, for example. In accordance with aspects of the present technique, the probe 14 may be configured to facilitate interventional procedures. In other words, in a presently contemplated configuration, the probe 14 may be configured to function as an invasive probe. It should also be noted that, although the embodiments illustrated are described in the context of a catheter-based probe, other types of probes such as endoscopes, laparoscopes, surgical probes, transrectal probes, transvaginal probes, intracavity probes, probes adapted for interventional procedures, or combinations thereof are also contemplated in conjunction with the present technique. Reference numeral 16 is representative of a portion of the probe 14 disposed inside the patient 12. Also, reference numeral 18 is indicative of a portion of the probe 14.

The system 10 may also include an imaging system 20 that is in operative association with the imaging catheter 14 and configured to facilitate acquisition of image data. It should be noted that although the exemplary embodiments illustrated hereinafter are described in the context of a medical imaging system, such as an ultrasound imaging system, other imaging systems and applications such as industrial imaging systems and non-destructive evaluation and inspection systems, such as pipeline inspection systems, liquid reactor inspection systems are also contemplated. Additionally, the exemplary embodiments illustrated and described hereinafter may find application in multi-modality imaging systems that employ ultrasound imaging in conjunction with other imaging modalities, position-tracking systems or other sensor systems.

Further, the imaging system 20 may be configured to display an image representative of a current position of the imaging catheter 14 within a region of interest in the patient 12. As illustrated in FIG. 1, the imaging system 20 may include a display area 22 and a user interface area 24. In accordance with aspects of the present technique, the display area 22 of the imaging system 20 may be configured to display the image generated by the imaging system 20 based on the image data acquired via the imaging catheter 14. Additionally, the display area 22 may be configured to aid the user in visualizing the generated image.

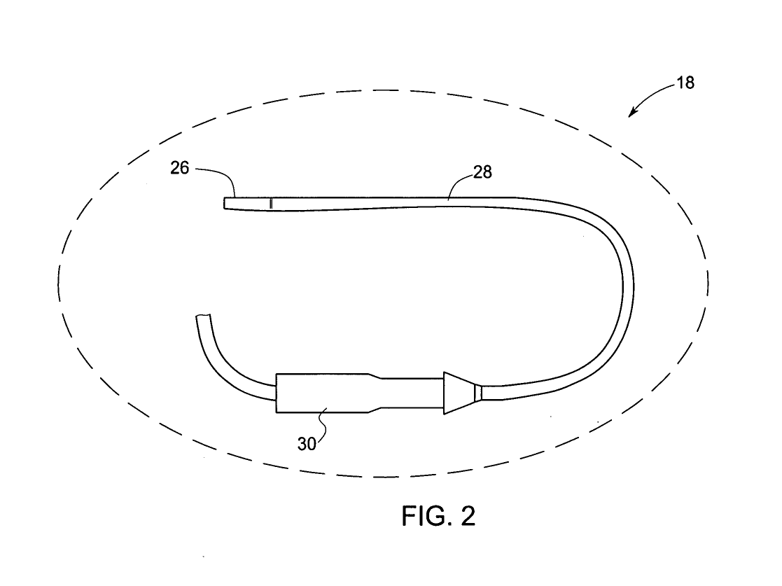

FIG. 2 illustrates an enlarged view of the portion 18 (see FIG. 1) of the imaging catheter 14 (see FIG. 1). As depicted in FIG. 2, a transducer assembly 26 configured for use in an invasive probe may be disposed on a distal end of a shaft 28. The imaging catheter 14 may also include a handle 30 configured to facilitate a user to manipulate the shaft 28. A distance between the transducer assembly 26 and the handle 30 may be in a range from about 10 cm to about 150 cm depending on the type of probe and application.

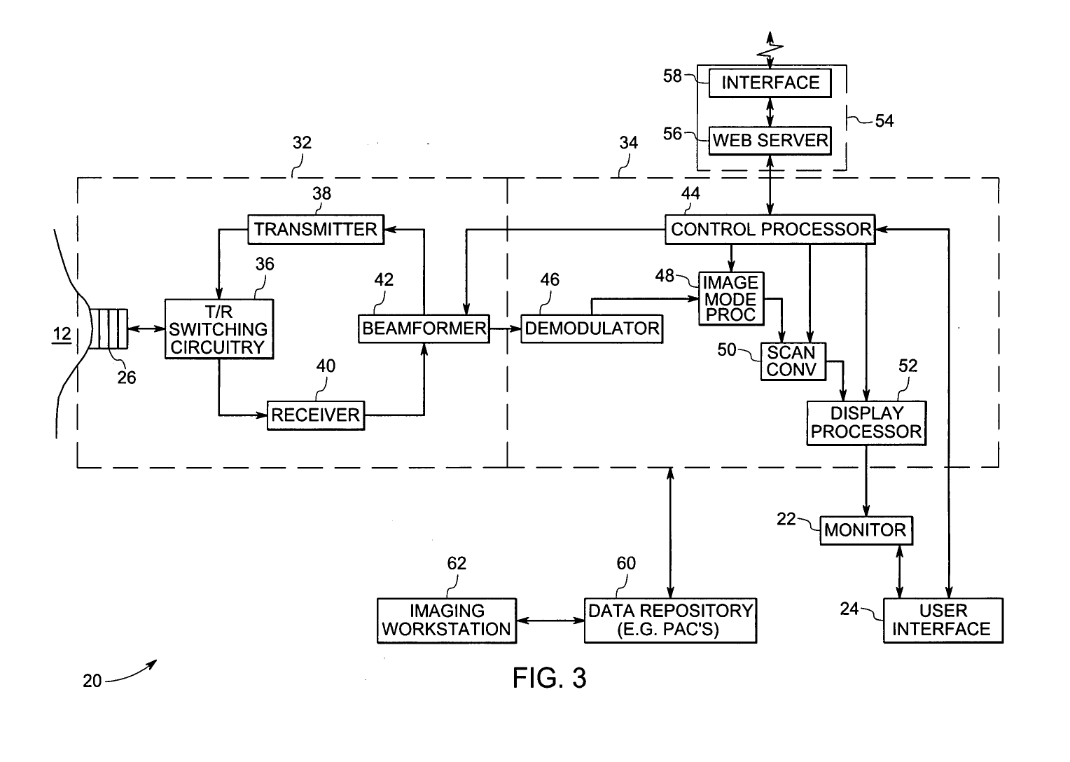

FIG. 3 is a block diagram of an embodiment of an ultrasound imaging system 20 depicted in FIG. 1. The ultrasound system 20 includes an acquisition subsystem 32 and a processing subsystem 34. The acquisition subsystem 32 may include a transducer assembly, such as the transducer assembly 26 (see FIG. 2). In addition, the acquisition subsystem includes transmit/receive switching circuitry 36, a transmitter 38, a receiver 40, and a beamformer 42. It may be noted that in a presently contemplated configuration, the transducer assembly 26 is disposed in the probe 14 (see FIG. 1). Also, in certain embodiments, the transducer assembly 26 may include a plurality of transducer elements (not shown) arranged in a spaced relationship to form a transducer array, such as a one-dimensional or two-dimensional transducer array, for example. Additionally, the transducer assembly 26 may include an interconnect structure (not shown) configured to facilitate operatively coupling the transducer array to an external device (not shown), such as, but not limited to, a cable assembly or associated electronics. In the illustrated embodiment, the interconnect structure may be configured to couple the transducer array to the T/R switching circuitry 36.

The processing subsystem 34 includes a control processor 44, a demodulator 46, an imaging mode processor 48, a scan converter 50 and a display processor 52. The display processor 52 is further coupled to a display monitor, such as the display area 22 (see FIG. 1), for displaying images. User interface, such as the user interface area 24 (see FIG. 1), interacts with the control processor 44 and the display monitor 22. The control processor 44 may also be coupled to a remote connectivity subsystem 54 including a web server 56 and a remote connectivity interface 58. The processing subsystem 34 may be further coupled to a data repository 60 configured to receive ultrasound image data. The data repository 60 interacts with an imaging workstation 62.

The aforementioned components may be dedicated hardware elements such as circuit boards with digital signal processors or may be software running on a general-purpose computer or processor such as a commercial, off-the-shelf personal computer (PC). The various components may be combined or separated according to various embodiments of the invention. Thus, those skilled in the art will appreciate that the present ultrasound imaging system 20 is provided by way of example, and the present techniques are in no way limited by the specific system configuration.

In the acquisition subsystem 32, the transducer assembly 26 is in contact with the patient 12 (see FIG. 1). The transducer assembly 26 is coupled to the transmit/receive (T/R) switching circuitry 36. Also, the T/R switching circuitry 36 is in operative association with an output of transmitter 38 and an input of the receiver 40. The output of the receiver 40 is an input to the beamformer 42. In addition, the beamformer 42 is further coupled to the input of the transmitter 38 and to the input of the demodulator 46. The beamformer 42 is also operatively coupled to the control processor 44 as shown in FIG. 3.

In the processing subsystem 34, the output of demodulator 46 is in operative association with an input of an imaging mode processor 48. Additionally, the control processor 44 interfaces with the imaging mode processor 48, the scan converter 50 and the display processor 52. An output of imaging mode processor 48 is coupled to an input of scan converter 50. Also, an output of the scan converter 50 is operatively coupled to an input of the display processor 52. The output of display processor 52 is coupled to the monitor 22.

The ultrasound system 20 transmits ultrasound energy into the patient 12 and receives and processes backscattered ultrasound signals from the patient 12 to create and display an image. To generate a transmitted beam of ultrasound energy, the control processor 44 sends command data to the beamformer 42 to generate transmit parameters to create a beam of a desired shape originating from a certain point at the surface of the transducer assembly 26 at a desired steering angle. The transmit parameters are sent from the beamformer 42 to the transmitter 38. The transmitter 38 uses the transmit parameters to properly encode transmit signals to be sent to the transducer assembly 26 through the T/R switching circuitry 36. The transmit signals are set at certain levels and phases with respect to each other and are provided to individual transducer elements of the transducer assembly 26. The transmit signals excite the transducer elements to emit ultrasound waves with the same phase and level relationships. As a result, a transmitted beam of ultrasound energy is formed in the patient 12 along a scan line when the transducer assembly 26 is acoustically coupled to the patient 12 by using, for example, ultrasound gel. The process is known as electronic scanning.

In one embodiment, the transducer assembly 26 may be a two-way transducer. When ultrasound waves are transmitted into a patient 12, the ultrasound waves are backscattered off the tissue and blood samples within the patient 12. The transducer assembly 26 receives the backscattered waves at different times, depending on the distance into the tissue they return from and the angle with respect to the surface of the transducer assembly 26 at which they return. The transducer elements convert the ultrasound energy from the backscattered waves into electrical signals.

The electrical signals are then routed through the T/R switching circuitry 36 to the receiver 40. The receiver 40 amplifies and digitizes the received signals and provides other functions such as gain compensation. The digitized received signals corresponding to the backscattered waves received by each transducer element at various times preserve the amplitude and phase information of the backscattered waves.

The digitized signals are sent to the beamformer 42. The control processor 44 sends command data to beamformer 42. The beamformer 42 uses the command data to form a receive beam originating from a point on the surface of the transducer assembly 26 at a steering angle typically corresponding to the point and steering angle of the previous ultrasound beam transmitted along a scan line. The beamformer 42 operates on the appropriate received signals by performing time delaying and focusing, according to the instructions of the command data from the control processor 44, to create received beam signals corresponding to sample volumes along a scan line within the patient 12. The phase, amplitude, and timing information of the received signals from the various transducer elements is used to create the received beam signals.

The received beam signals are sent to the processing subsystem 34. The demodulator 46 demodulates the received beam signals to create pairs of I and Q demodulated data values corresponding to sample volumes along the scan line. Demodulation is accomplished by comparing the phase and amplitude of the received beam signals to a reference frequency. The I and Q demodulated data values preserve the phase and amplitude information of the received signals.

The demodulated data is transferred to the imaging mode processor 48. The imaging mode processor 48 uses parameter estimation techniques to generate imaging parameter values from the demodulated data in scan sequence format. The imaging parameters may include parameters corresponding to various possible imaging modes such as B-mode, color velocity mode, spectral Doppler mode, and tissue velocity imaging mode, for example. The imaging parameter values are passed to the scan converter 50. The scan converter 50 processes the parameter data by performing a translation from scan sequence format to display format. The translation includes performing interpolation operations on the parameter data to create display pixel data in the display format.

The scan converted pixel data is sent to the display processor 52 to perform any final spatial or temporal filtering of the scan converted pixel data, to apply grayscale or color to the scan converted pixel data, and to convert the digital pixel data to analog data for display on the monitor 22. The user interface 24 is coupled to the control processor 44 to allow a user to interface with the ultrasound system 20 based on the data displayed on the monitor 22.

Currently available transducer assemblies typically include one or more transducer elements, one or more matching layers, and a lens. The transducer elements may be arranged in a spaced relationship, such as, but not limited to, an array of transducer elements disposed on a layer, where each of the transducer elements may include a transducer front face and a transducer rear face. As will be appreciated by one skilled in the art, the transducer elements may be fabricated employing materials, such as, but not limited to lead zirconate titanate (PZT), polyvinylidene difluoride (PVDF) or composite PZT. The transducer assembly may also include one or more matching layers disposed adjacent to the front face of the array of transducer elements, where each of the matching layers may include a matching layer front face and a matching layer rear face. The matching layers facilitate matching of an impedance differential that may exist between the high impedance transducer elements and a low impedance patient 12 (see FIG. 1). The lens may be disposed adjacent to the matching layer front face and provides an interface between the patient 12 and the matching layer.

Additionally, the transducer assembly may include a backing structure, having a front face and a rear face, which may be fabricated employing a suitable acoustic damping material possessing high acoustic losses. The backing structure may be acoustically coupled to the rear face of the array of transducer elements, where the backing structure facilitates the attenuation of acoustic energy that may emerge from the rear face of the array of transducer elements. In addition, the backing structure may include an interconnect structure. Moreover, the transducer assembly may also include an electrical shield (not shown) that facilitates the isolation of the transducer elements from the external environment. The electrical shield may include metal foils, where the metal foils may be fabricated employing metals such as, but not limited to, copper, aluminum, brass, or gold.

As previously discussed, it may be desirable to enhance the imaging performance of the transducer assembly by increasing an elevational aperture of the probe. More particularly, it may be desirable to develop a transducer assembly that advantageously maximizes elevational aperture size, thereby resulting in enhanced image resolution and sensitivity of the probe. The exemplary transducer assembly will be described in greater detail hereinafter.

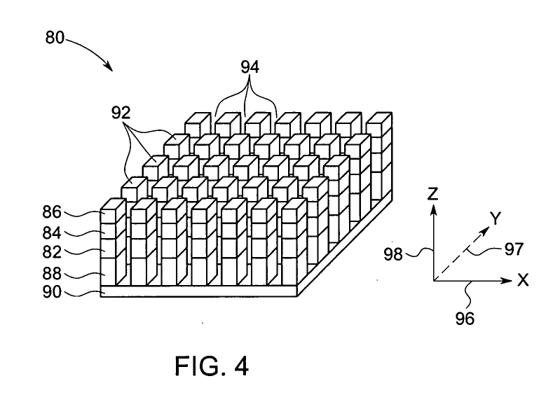

Referring now to FIG. 4, a perspective view of an exemplary embodiment 80 of a transducer assembly is illustrated. In a presently contemplated configuration, the transducer assembly 80 is shown as including an acoustic layer 82 having a first side and a second side, where the second side is opposite the first side. In one embodiment, the first side may include a top side and the second side may include a bottom side. As will be appreciated, the acoustic layer 82 may be configured to generate and transmit acoustic energy into the patient 12 (see FIG. 1) and receive backscattered acoustic signals from the patient 12 to create and display an image. In addition, the acoustic layer 82 may include a plurality of transducer elements. Furthermore, the acoustic layer 82 may include lead zirconate titanate (PZT), a piezoelectric ceramic, a piezocomposite, a piezoelectric single crystal, or a piezopolymer. It may be noted that in certain embodiments, the acoustic layer 82 may include multiple layers of the aforementioned materials. More particularly, in one embodiment, the acoustic layer 82 may include multiple layers of the same material, while in another embodiment, the acoustic layer 82 may include multiple layers of different materials. Also, the acoustic layer 82 may have a thickness in a range from about 50 microns to about 600 microns. In one embodiment, the acoustic layer 82 may have a thickness of about 65 microns.

In accordance with aspects of the present technique, the transducer assembly 80 may include at least one matching layer disposed on the first side of the acoustic layer 82. It may be noted that the at least one matching layer may be configured to have an acoustic impedance less than the acoustic impedance of the acoustic layer 82. For example, the acoustic impedance of the at least one matching layer may be in a range from about 4 MRayls to about 15 MRayls, while the acoustic impedance of the acoustic layer 82 may be in a range from about 10 MRayls to about 35 MRayls.

In one embodiment, a first matching layer 84, itself having a top side and a bottom side may be disposed on the first side of the acoustic layer 82. As will be appreciated, the first matching layer 84 may be configured to facilitate the matching of an impedance differential that may exist between the high impedance transducer elements and a low impedance patient 12. In a presently contemplated configuration, the first matching layer 84 may include filled epoxy, metal-impregnated graphite, or glass ceramics. In accordance with aspects of the present technique, the first matching layer 84 may have a thickness in a range from about 40 microns to about 300 microns. In one embodiment, the first matching layer 84 may have a thickness of about 80 microns.

In a presently contemplated configuration, the transducer assembly 80 may also include a second matching layer 86 having a top side and a bottom side disposed on the top side of the first matching layer 84. As noted with respect to the first matching layer 84, the second matching layer 86 may also be configured to facilitate the matching of an impedance differential that may exist between the high impedance transducer elements and a low impedance patient 12. Also, as previously noted with reference to the first matching layer 84, in a presently contemplated configuration, the second matching layer 86 may include unfilled epoxy or plastic, such as polysulphone or polystyrene. Furthermore, the second matching layer 86 may have a thickness in a range from about 30 microns to about 250 microns. In certain embodiments, the second matching layer 86 may have a thickness of about 80 microns.

According to exemplary embodiments of the present technique, the transducer assembly 80 may include a dematching layer 88 disposed adjacent the bottom side of the acoustic layer 82. In one embodiment, the dematching layer 88 may be disposed on the bottom side of the acoustic layer 82, for example. This dematching layer 88 may be constructed employing a material having a high impedance. It may be noted that the acoustic impedance of the dematching layer 88 may be configured to be substantially higher than the acoustic impedance of the acoustic layer 82. For example, the acoustic impedance of the acoustic layer 82 may be in a range from about 10 MRayls to about 35 MRayls, while the acoustic impedance of the dematching layer 88 may be in a range from about 40 MRayls to about 100 MRayls. In certain embodiments, the high impedance material may include tungsten, for example.

According to aspects of the present technique, the dematching layer 88 may be configured to be about one-fourth of a wavelength thick at an operating frequency of the transducer. The dematching layer 88 may be configured to function as an acoustic impedance transformer, dramatically increasing the effective acoustic impedance of the material on a rear face (i.e., away from the acoustic layer 82) of the dematching layer 88 to a value substantially greater than the impedance of the acoustic layer 82. Consequently, a majority of the acoustic energy is reflected out a front face of the acoustic layer 82. However, the dematching layer 88 may be configured to include relatively thinner layers such as layers having a thickness of about one-sixth of a wavelength, for example. It may be noted that in certain embodiments, the dematching layer 88 may also be configured to have a thickness of about one-third of a wavelength, while in certain other embodiments, the dematching layer 88 may be configured to have a thickness of about one-eighth of a wavelength. Accordingly, the dematching layer 88 may be configured to have a thickness in a range from about 50 microns to about 500 microns. In certain embodiments, the dematching layer 88 may be configured to have a thickness of about 230 microns. It may be noted that for the dematching layer 88 having an impedance of about 100 MRayls and a thickness of about one-fourth wavelength, the effective impedance seen to the rear of the acoustic layer 82 and towards the dematching layer 88 is about 24,000,000 MRayls for an air-backed transducer assembly where air is present to the rear of the dematching layer 88. In a similar fashion, the effective impedance seen to the rear of the acoustic layer 82 and towards the dematching layer 88 is about 6,667 MRayls for a water-backed transducer assembly where water is present to the rear of the dematching layer 88. Consequent to the extreme impedance mismatch between the acoustic layer 82 and the effective impedance to the rear of the acoustic layer 82, a majority of the acoustic energy is reflected towards the front surface of the acoustic layer 82.

The relatively higher impedance of the dematching layer 88 relative to the impedance of the acoustic layer 82 results in the acoustic layer 82 operating in a quarter-wavelength resonance mode, instead of a half-wave resonance mode as is the case for transducers with conventional low impedance backing layers. Consequently, employing the exemplary transducer assembly 80 having a dematching layer 88, for a given operating frequency, the acoustic layer 82 may be configured to have a thickness that is about half the thickness of an acoustic layer employed in conventional stacks. For example, for a given operating frequency, the thickness of the acoustic layer 82 in the present exemplary transducer assembly 80 may be about 65 microns as opposed to an acoustic layer having a thickness of about 130 microns in a conventional transducer assembly having a low impedance backing layer. As will be appreciated, currently available transducer assemblies typically include a backing layer. However, in accordance with exemplary aspects of the present technique, no such backing layer is provided in the arrangement illustrated in FIG. 4. More particularly, the exemplary embodiment of the transducer assembly 80 illustrated in FIG. 4 does not include a backing layer that is highly attenuative. It may be noted that a backing layer that is highly attenuative may be defined as a backing layer that has an acoustic attenuation that is relatively greater than about 30 dB total round-trip attenuation at the center frequency of operation.

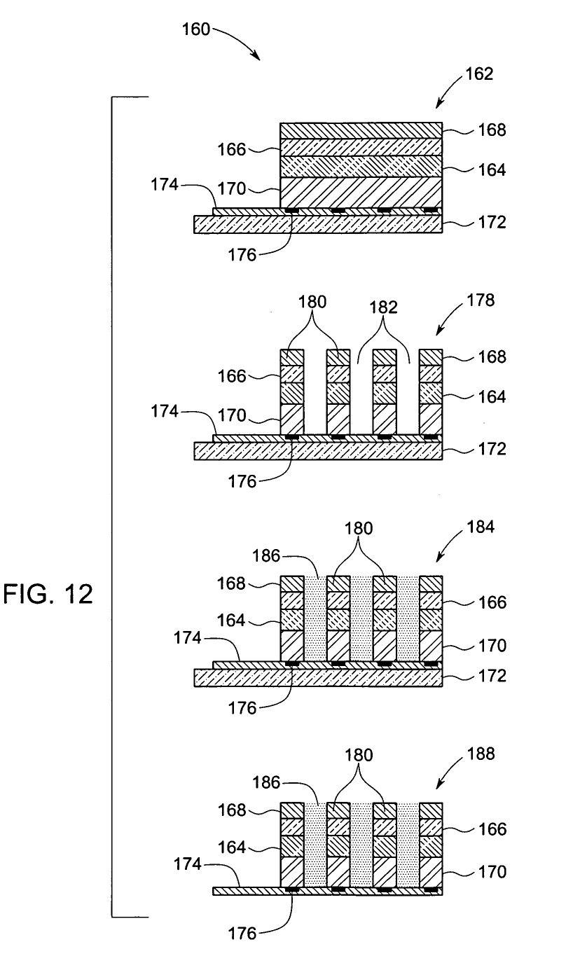

Additionally, the transducer assembly 80 may include an interconnect layer 90 that may be configured to operatively couple the acoustic layer of the transducer assembly 80 to a cable assembly (not shown) or electronics (not shown). The interconnect layer 90 may include a flexible interconnect layer that includes at least one conductive element disposed on a flexible substrate, where at least one conductive element may be configured to facilitate coupling the plurality of transducer elements to a cable assembly, for example. In the embodiment illustrated in FIG. 4, the interconnect layer 90 is shown as being disposed adjacent to the dematching layer 88. However, the interconnect layer 90 may be disposed at different positions within the transducer assembly 80 and will be described with reference to FIGS. 12-14.

With continuing reference to FIG. 4, reference numeral 92 is representative of a plurality of transducer elements, while reference numeral 94 is used to represent inter-element space. In addition, reference numerals 96, 97 and 98 may be representative of a X-direction, a Y-direction, and a Z-direction respectively.

It may be noted that, in accordance with exemplary aspects of the present technique, the transducer assembly 80 may not include a highly attenuative backing layer otherwise present in a conventional transducer assembly. As will be appreciated, the low impedance backing layer in a conventional transducer assembly may be configured to serve a structural function and/or an acoustic function. The backing layer may be configured to provide support to a transducer array that may be built thereon. In certain other embodiments, the backing layer may be configured to facilitate attenuation of acoustic energy that may emerge from an array of transducer elements. Furthermore, the low impedance backing layer employed in a conventional transducer assembly may have a typical thickness of about 800 microns or more. Consequently, if the transducer assembly includes a backing layer, the effective thickness of the transducer assembly may be substantially increased. In a space-constrained application, such as a catheter, this increased thickness impedes fitting of the array within the widest portion of the catheter, thereby resulting in a reduced elevational aperture, which in turn results in reduced resolution and sensitivity of the transducer assembly.

By implementing the transducer assembly 80 as described hereinabove, the thickness of the transducer assembly 80 may be reduced. Furthermore, in one embodiment, the thickness of the transducer assembly 80 having the dematching layer 88 may be reduced by one half as compared to the thickness of a comparable conventional transducer assembly having a low impedance backing layer. Consequent to the reduction in thickness of the transducer assembly 80, the width of the transducer assembly 80 may be accordingly increased thereby resulting in a transducer assembly 80 having a larger elevational aperture. Also, additional space savings within a catheter lumen may advantageously be obtained.

Further, the transducer assembly 80 illustrated in FIG. 4 that may be configured for use in an intra-vascular ultrasound (IVUS) catheter is also contemplated in accordance with further aspects of the present technique. As will be appreciated, the IVUS catheters may have a diameter of about 1 mm and may be configured to fit within the coronary arteries. Also, the transducer assembly configured for use in IVUS catheters may be configured to operate in a range from about 15 MHz to about 50 MHz. As will be appreciated, the thickness of the acoustic layer varies inversely with a desired frequency. Accordingly, the acoustic layer 82 may have thickness in a range from about 20 microns to about 80 microns. For example, in one embodiment, a transducer assembly configured to operate at 50 MHz may include an acoustic layer having a thickness of about 20 microns, while an acoustic layer having a thickness of about 80 microns may be employed in a transducer assembly configured to operate at about 15 MHz. The first matching layer 84 may have a thickness in a range from about 20 microns to about 80 microns, while the second matching layer 86 may have a thickness in a range from about 15 microns to about 60 microns. Additionally, the dematching layer 88 may have a thickness in a range from about 20 microns to about 90 microns.

It may be noted that the corresponding range of thicknesses of each of the acoustic layer 82, the first matching layer 84, the second matching layer 86 and the dematching layer 88 may be adjusted according to the application that entails the use of the transducer assembly 80. More particularly, different applications of the transducer assembly 80 may call for diverse range of frequencies of operation. The range of thickness of each of the constituent layers 82, 84, 86, 88 of the transducer assembly 80 may accordingly be adjusted based upon the application that involves use of the transducer assembly 80.

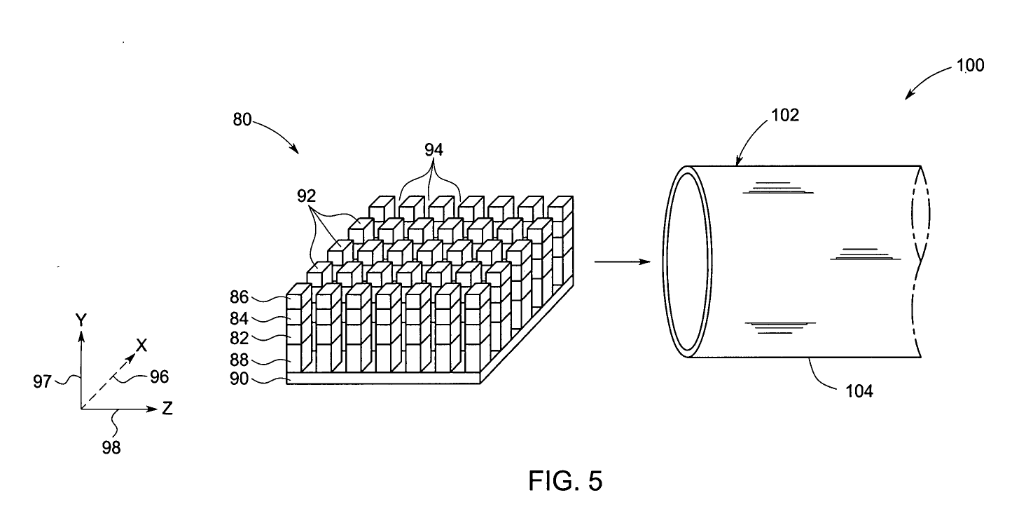

FIG. 5 illustrates an exemplary method 100 for forming a probe having an exemplary transducer assembly, such as the transducer assembly illustrated in FIG. 4, in accordance with aspects of the present technique. In certain embodiments, the invasive probe may include an imaging catheter, an endoscope, a laparoscope, a surgical probe, a transrectal probe, a transvaginal probe, an intracavity probe, or a probe adapted for interventional procedures, as previously noted. Reference numeral 80 is representative of a transducer assembly illustrated in FIG. 4. As previously described, the transducer assembly 80 may be formed by disposing a first matching layer 84 on a first side of an acoustic layer 82 and a second matching layer 86 on a first side of the first matching layer 84, in one embodiment. Furthermore, in certain embodiments, a high impedance dematching layer 88 may be disposed on a second side of the acoustic layer 82. Additionally, an interconnect layer 90 may be disposed adjacent to the dematching layer 88, in one embodiment.

In certain embodiments, following construction of the transducer assembly 80, the transducer assembly 80 may be disposed in a probe 102, as illustrated in FIG. 5. It may be noted that the invasive probe 102 may include an outer envelope 104 sized and configured to be disposed within an anatomical region. Accordingly, the transducer assembly 80 may be disposed in the outer envelope 104 of the invasive probe 102.

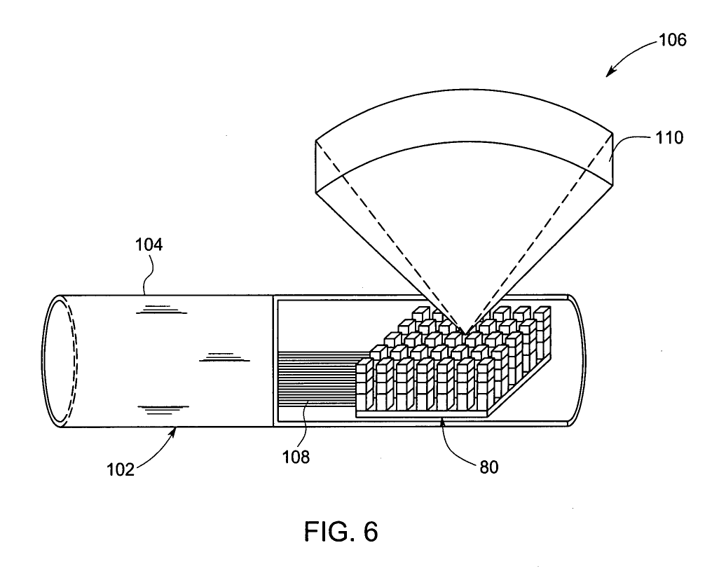

FIG. 6 is a perspective view 106 of a side viewing probe 102 including the transducer assembly 80 having the exemplary dematching layer 88. Reference numeral 108 is representative of an interconnect that may be configured to operatively couple the acoustic layer 82 of the transducer assembly 80 to a cable assembly (not shown) or electronics (not shown). Also, a side viewing imaging volume of the side viewing probe 102 may be generally represented by reference numeral 110.

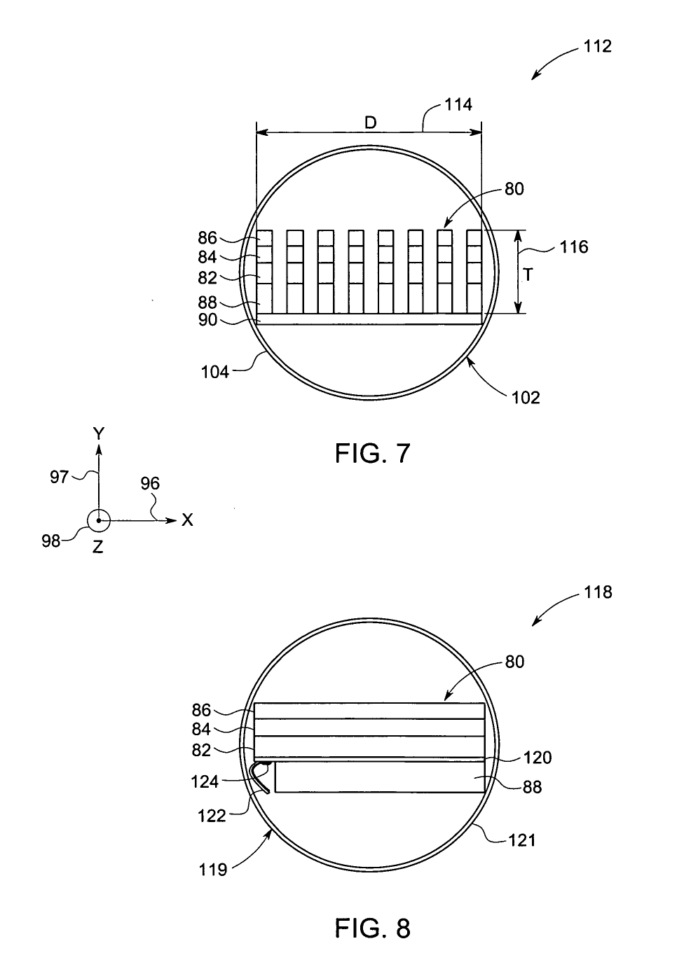

Referring now to FIG. 7, an end view 112 is shown of the invasive probe 102 including the transducer assembly 80 having the dematching layer 88 illustrated in FIG. 6. In the illustrated embodiment, reference numeral 114 is representative of an elevational aperture of the transducer assembly 80. Additionally, a thickness of the transducer assembly 80 may be represented by reference numeral 116.

As before, here again, by implementing the transducer assembly 80 as described hereinabove, the transducer assembly 80 having the dematching layer 88 may be configured to have a substantially reduced thickness 116 as opposed to the thickness of a conventional transducer assembly having a low impedance backing layer. For example, a typical thickness of a conventional transducer assembly (not shown) including a low impedance backing layer having a thickness of about 800 microns may be about 1090 microns. However, a typical thickness of the exemplary transducer assembly 80 including the high impedance dematching layer 88 having a thickness of about 230 microns is about 455 microns. It may be noted that the effective thickness of the transducer assembly 80 having the dematching layer 88 may be reduced by a factor of at least two as compared to the effective thickness of the conventional transducer assembly having the low impedance backing layer. Moreover, for a given operating frequency, the thickness of the acoustic layer 82 in the exemplary transducer assembly 80 may be advantageously reduced when compared with the thickness of an acoustic layer in a conventional transducer assembly having a low impedance backing layer, thereby resulting in a reduction in the overall thickness of the transducer assembly 80. Consequently, the elevational aperture 114 of the exemplary transducer assembly 80 may be substantially enhanced, thereby advantageously resulting in enhanced image contrast for one-dimensional arrays and enhanced image resolution for two-dimensional arrays as well as improved sensitivity of the invasive probe 102.

FIG. 8 illustrates an end view 118 of an invasive probe 119 having an outer envelope 121 depicting a mode of operatively coupling a flex circuit to the acoustic layer 82. It may be noted that in FIG. 8, for simplicity of illustration a one-dimensional (1D) array is illustrated as opposed to a two-dimensional (2D) array. In the illustrated embodiment, reference numeral 120 embodies a bottom electrode associated with the acoustic layer 82. In addition, reference numeral 122 is representative of a flex circuit configured to operatively couple the acoustic layer 82 to a cable assembly (not shown) or electronics (not shown), for example. Moreover, an electrical connection between the bottom electrode 120 and the flex circuit 122 is represented by reference numeral 124.

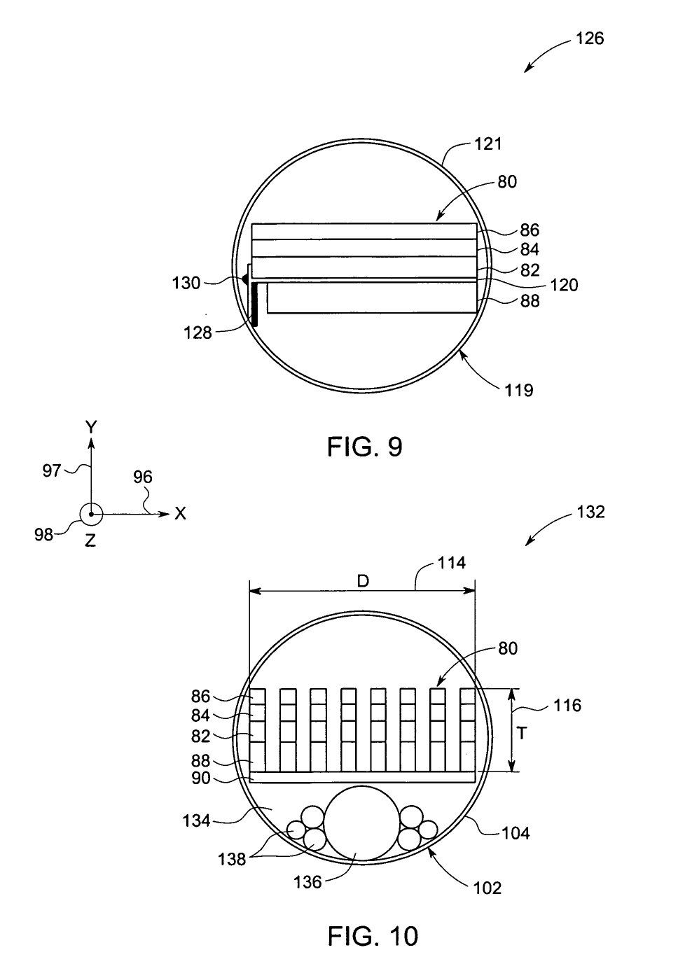

Turning now to FIG. 9, an end view 126 is shown of the invasive probe 119 depicting an alternate mode of operatively coupling a flex circuit to the acoustic layer 82. As noted hereinabove with reference to FIG. 8, for simplicity of illustration a one-dimensional (1D) array is illustrated in FIG. 9 as opposed to a two-dimensional (2D) array. In the illustrated embodiment, reference numeral 128 embodies a flex circuit configured to operatively couple the acoustic layer 82 to a cable assembly (not shown) or electronics (not shown), for example. Additionally, an electrical coupling between the bottom electrode 120 and the flex circuit 128 is represented by reference numeral 130.

FIG. 10 is an end view 132 of the invasive probe 102 illustrated in FIG. 6 depicting additional components disposed within the invasive probe 102. It may be noted that the invasive probe 102 may include the exemplary low-profile transducer assembly 80 (see FIG. 4) disposed in the outer envelope 104. As previously noted, use of the high impedance dematching layer 88 advantageously results in the transducer assembly 80 having a relatively smaller effective thickness, and therefore an enhanced elevational aperture. In other words, the transducer assembly 80 has a relatively thinner profile. Consequently, the low-profile of the transducer assembly 80 results in additional room inside the probe lumen 134. As a result, other components, such as, but not limited to, a working port, a fluid passageway, electrical leads, or combinations thereof, may be disposed within the probe lumen 134 of the invasive probe 102. In the illustrated embodiment, the invasive probe 102 is shown as including a working port 136 and a plurality of electrical leads 138 in addition to the low-profile transducer assembly 80.

In one embodiment, the working port 136 may be configured to run the entire length of the probe 102. Also, the working port 136 may provide an additional lumen within the probe lumen 134. Furthermore, the working port 136 may be configured to facilitate delivery of therapy to one or more regions of interest. As used herein, “therapy” is representative of delivery of tools, such as needles for delivering gene therapy, for example. Additionally, as used herein, “delivering” may include various means of providing therapy to the one or more regions of interest, such as conveying therapy to the one or more regions of interest or directing therapy towards the one or more regions of interest. Also, the electrical leads 138 may be employed to facilitate connection to additional sensors, such as electrophysiological sensors, temperature sensors, pressure sensors and/or position sensors. Alternatively, the electrical leads 138 may be utilized to connect to a motor, where the motor may be configured to rotate the transducer array in an oscillatory manner for four-dimensional (4D) imaging.

In accordance with aspects of the present technique, the probe lumen 134 may also include additional ports (not shown). For example, the additional port may include a fluid passageway. Also, in certain embodiments, the additional ports, such as the fluid passageway, may be configured to facilitate delivery of fluids, such as therapeutic drugs, imaging contrast agents, etc., to one or more regions of interest, while in certain other embodiments, the additional ports may be configured to facilitate passage of a guide wire and/or optic fibers.

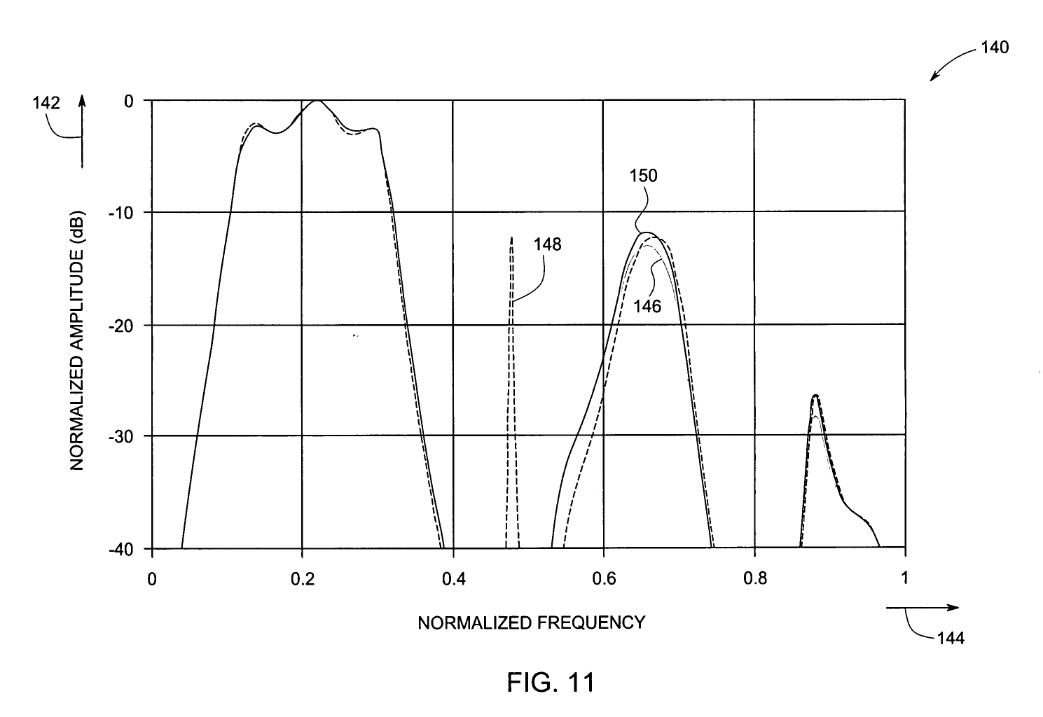

FIG. 11 is a graphical representation of exemplary simulation results depicting the effect of various materials disposed to the rear of a dematching layer in the low-profile transducer assembly (illustrated in FIG. 4), in accordance with aspects of the present technique. In FIG. 11, a graphical representation of simulation results 140 depicting a variation in amplitude 142 is plotted against a variation in normalized frequency 144.

Response curve 146 represents a variation of the amplitude 142 as a function of the normalized frequency 144 for the case where a material that is disposed to the rear of the dematching layer 88 (see FIG. 4), in the low-profile transducer assembly 80 (see FIG. 4), includes an acoustically attenuating backing material.

Additionally, response curve 148 embodies a variation of the amplitude 142 as a function of the normalized frequency 144 for the case where a material that is disposed to the rear of the dematching layer 88 in the low-profile transducer assembly 80 includes a polymer layer which has air on the rear face (i.e., away from dematching layer 88). In certain embodiments, the polymer layer may include an interconnect layer.

Furthermore, response curve 150 is representative of a variation of the amplitude 142 as a function of the normalized frequency 144 for the case where no additional layer is disposed to the rear of the dematching layer 88 in the low-profile transducer assembly 80. In other words, the dematching layer 88 in the transducer assembly 80 may be configured to be in contact with air, for example.

As may be seen from the graphical representation of exemplary simulation results illustrated in FIG. 11, when the transducer assembly includes a dematching layer, replacing the conventional acoustically attenuating backing layer with a polymer layer or air, in accordance with the technique as described hereinabove, will have minimal impact on the frequency response of the transducer assembly 80. In addition, an extra resonance appears when air is behind the polymer layer, as may be seen in FIG. 11. This mode is a quarter-wave resonance of the polymer layer and may be adjusted so that this undesirable resonance lies outside the frequency band of interest by altering the thickness of the polymer layer.

Turning now to FIG. 12, progressive structures are illustrated, made in an exemplary process 160 of fabricating an exemplary low-profile transducer assembly, such as the low-profile transducer assembly 80 shown in FIG. 4, in accordance with aspects of the present technique. As previously noted, the low-profile transducer assembly may include an acoustic layer, at least one matching layer disposed on a first side of the acoustic layer and a dematching layer disposed on a second side of the acoustic layer, where the second side of the acoustic layer is opposite the first side of the acoustic layer.

The process begins at step 162 where an exemplary acoustic stack is formed. In accordance with aspects of the present technique, the process of forming a transducer assembly, such as the transducer assembly 80 (see FIG. 4), may include forming an acoustic layer 164 having a top side and a bottom side. Electrodes may be sputtered and/or plated on the top and bottom sides of the acoustic layer 164. As will be appreciated, the electrodes may have different physical configurations, particularly for ground and signal electrodes. In one embodiment, the electrodes may include a wrap-around configuration. The acoustic layer 164 may be configured to have a thickness in a range from about 50 microns to about 600 microns.

Following formation of the acoustic layer 164, a first matching layer 166 having a top side and a bottom side may be disposed on the top side of the acoustic layer 164. The first matching layer 166 may be configured to have a thickness in a range from about 40 microns to about 300 microns. Subsequently, a second matching layer 168 having a top side and a bottom side may be disposed on the top side of the first matching layer 166. As described with respect to the first matching layer 166, the second matching layer 168 may be configured to have a thickness in a range from about 30 microns to about 250 microns. The first and second matching layers 166, 168 may be configured to facilitate the matching of an impedance differential that may exist between the high impedance transducer elements and a low impedance patient 12 (see FIG. 1). It may be appreciated that such transducers may include a single or multiple matching layers. Currently available transducers typically employ two matching layers, where the use of two matching layers in the transducers may represent the best trade-off between performance and stack thickness for space-constrained applications such as catheters.

Additionally, at step 162, an exemplary dematching layer 170 having a top side and a bottom side may be disposed on the bottom side of the acoustic layer 164. In other words, the dematching layer 170 may be disposed on a side of the acoustic layer 164 that is opposite the side that the first matching layer 166 is disposed on. Furthermore, the dematching layer 170 may be configured to have a thickness in a range from about 50 microns to about 500 microns. Moreover, as will be appreciated, the dematching layer 170 may be configured to be electrically conductive. As previously described, the effective thickness of the dematching layer 170 may be substantially less than the thickness of a low-impedance acoustic backing layer, thereby advantageously resulting in a transducer assembly having a low profile, which advantageously permits increasing the width of the acoustic layer, resulting in an enhanced elevational aperture. A low-profile transducer assembly may thus be formed by stacking the second matching layer 168, the first matching layer 166, the acoustic layer 164 and the dematching layer 170 and bonding the layers.

With continuing reference to step 162, substrate 172 having a top side and a bottom side may be selected. The substrate 172 may include one of a plastic, a metal, a ceramic, silicon, a polymer or glass. It may be noted that the substrate 172 may be configured to provide mechanical strength to the transducer assembly during the fabrication process. In addition, at step 162, an interconnect layer 174 having a top side and a bottom side may be disposed on the top side of the substrate 172. In accordance with aspects of the present technique, the interconnect layer 174 may include a single layer interconnect circuit or a multi-layer interconnect circuit. As will be appreciated, the interconnect layer 174 may be configured to operatively couple the transducer elements to a cable assembly, for example. Alternatively, the substrate 172 and the interconnect layer 174 may be the same piece, where conductive elements are disposed directly on the substrate 172 or are internal to the substrate 172.

Additionally, in step 162, the acoustic stack having the electrically conductive dematching layer 170, the acoustic layer 164, and the first and second matching layers 166, 168 may be operatively coupled to the interconnect layer 174, in certain embodiments. Alternatively, the acoustic stack may be operatively coupled to the substrate 172. However, in certain other embodiments, the acoustic stack may be operatively coupled to both the interconnect layer 174 and the substrate 172. The methods of electrically coupling the transducer assembly to the interconnect layer 174 and the substrate 172 may include lamination with electrically conductive or non-conductive epoxy, for example. Moreover, reference numeral 176 is representative of an electrical connection.

Step 178 depicts dicing of the transducer assembly to form a plurality of transducer elements. Accordingly, one or more saw kerfs 182 may extend through the four layers of the transducer assembly, where the four layers include the second matching layer 168, the first matching layer 166, the acoustic layer 164 and the dematching layer 170. In accordance with further aspects of the present technique, the one or more saw kerfs 182 may also partially extend into the interconnect layer 174. Consequent to the dicing of the transducer assembly at step 178, a plurality of transducer elements 180 may be formed.

Further, at step 184, a kerf filler material 186 may be disposed in the inter-element spaces 182 between the plurality of transducer elements 180. The kerf filler 186 may include filled or unfilled silicone or epoxy. Also, the kerf filler 186 may be configured to mechanically strengthen the transducer assembly by filling the inter-element space 182 thereby resulting in a less fragile and more reliable assembly. The kerf filler 186 may be configured to have low shear stiffness or high shear attenuation, thereby resulting in minimized inter-element cross talk. Following step 184, the substrate 172 may be removed, at step 188. Techniques, such as, but not limited to, chemical etching, mechanical grinding, or thermal methods may be employed to remove the substrate 172, at step 188. It may be noted that, in accordance with aspects of the present technique, step 184 may be an optional step in the process of forming the transducer assembly. Furthermore, in certain embodiments, step 188 that involves the removal of the substrate, may also be an optional step.

Additionally, electrical ground connections in the transducer assembly may be accomplished via use of a relatively thin foil (not shown), where the relatively thin foil may be laminated to the top of the second matching layer 168, in one embodiment. It may be noted that in certain embodiments both the first matching layer 166 and the second matching layer 168 may be conductive or have micro-vias (not shown) disposed through them to facilitate the ground connections. Alternatively, the first matching layer 166 may be conductive, while one or more micro-vias may be disposed through the second matching layer 168, where the micro-vias may be filled with epoxy (not shown). In certain other embodiments, the ground connections may be accomplished using micro-vias and/or traces that may be disposed along the sides of the individual transducer elements 180 and coupled to pads on the interconnect layer 174.

By employing the method of forming the transducer assembly as described hereinabove, a low-profile transducer assembly may be obtained. As previously noted, the low-profile transducer assembly advantageously results in enhanced resolution and improved sensitivity. Also, the low-profile transducer assembly thus formed may then be disposed in an invasive probe sized and configured for insertion in an anatomical region thus facilitating formation of an invasive probe with enhanced imaging resolution and sensitivity.

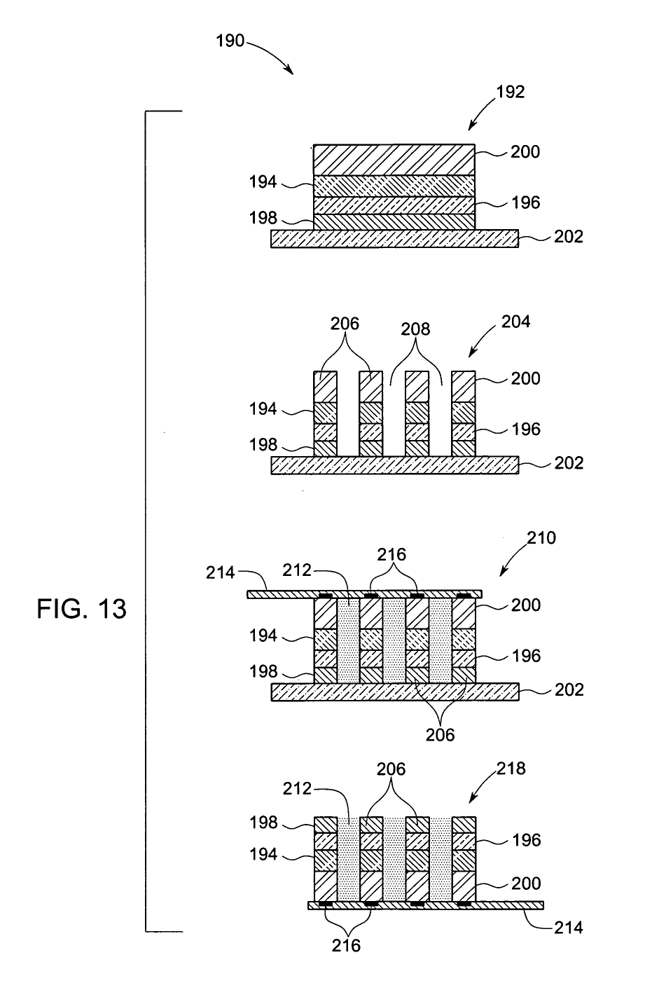

FIG. 13 represents another series of structures made by another exemplary method 190 for forming a low-profile transducer assembly, in accordance with aspects of the present technique. Step 192 represents an initial step where an exemplary transducer assembly is formed. In accordance with aspects of the present technique, the process of forming a transducer assembly, such as the transducer assembly 80 (see FIG. 4), may include forming an acoustic layer 194 having a top side and a bottom side. As previously noted, the acoustic layer 194 may include a PZT ceramic, a piezoelectric ceramic, a piezocomposite, a piezoelectric single crystal, or a piezopolymer. In addition, the acoustic layer 194 may be configured to have a thickness in a range from about 50 microns to about 600 microns, as previously noted. Electrodes may be sputtered and/or plated on the top and bottom sides of the acoustic layer 194.

Subsequently, a first matching layer 196 having a top side and a bottom side may be disposed on the bottom side of the acoustic layer 194. As previously noted, the first matching layer 196 may be configured to have a thickness in a range from about 40 microns to about 300 microns. A second matching layer 198 having a top side and a bottom side may then be disposed on the bottom side of the first matching layer 196. In certain embodiments, the second matching layer 198 may be configured to have a thickness in a range from about 30 microns to about 250 microns.

Additionally, at step 192, an exemplary dematching layer 200 having a top side and a bottom side may be disposed on the top side of the acoustic layer 194. In other words, the dematching layer 200 may be disposed on a side of the acoustic layer 194 that is opposite the side of the acoustic layer 194 that the first matching layer 196 is disposed on. Furthermore, the dematching layer 200 may be configured to have a thickness in a range from about 50 microns to about 500 microns. As previously noted, the dematching layer 200 may be configured to be electrically conductive.

Furthermore, at step 192, a substrate 202 having a top side and a bottom side may be selected. The substrate 202 may be configured to facilitate providing mechanical strength to the transducer assembly during the fabrication process. Also, the substrate 202 may include one of a plastic, a metal, a ceramic, silicon, a polymer or glass.

With continuing reference to step 192, the acoustic stack including the dematching layer 200, the acoustic layer 194, the first matching layer 196 and the second matching layer 198 may be disposed on the top side of the substrate 202 such that the bottom side of the second matching layer 198 is operatively coupled to the top side of the substrate 202. In other words, the transducer assembly may be bonded together on the substrate 202 in an “upside-down” configuration, with the second matching layer 198 in contact with the substrate 202.

Subsequently, at step 204, the transducer assembly may be diced from a backside of the acoustic stack to form a plurality of transducer elements 206. Reference numeral 208 is representative of one or more saw kerfs that may extend through the four layers of the transducer assembly, where the four layers include the dematching layer 200, the acoustic layer 194, the first matching layer 196 and the second matching layer 198. In accordance with further aspects of the present technique, the one or more saw kerfs 208 may extend either partially or fully through the second matching layer 198. Further, in certain embodiments, the saw kerfs 208 may extend partially into the substrate 202.

Following step 204, a kerf filler 212 may optionally be disposed in the inter-element space 208 between the plurality of transducer elements 206, at step 210. As previously noted with reference to FIG. 12, the kerf filler 212 may be configured to mechanically strengthen the transducer assembly, thereby creating a less fragile and more reliable assembly. The kerf filler 212 may be configured to have low shear stiffness or high shear attenuation, thereby resulting in reduced inter-element cross talk. Furthermore, at step 210, an interconnect layer 214 having a top side and a bottom side may be disposed on the top side of the dematching layer 200 such that the bottom side of the interconnect layer 214 is in operative association with the top side of the dematching layer 200. Alternatively, the interconnect layer 214 may be part of an initial lamination. As previously noted, the interconnect layer 214 may include a single layer interconnect circuit or a multi-layer interconnect circuit. Reference numeral 216 is representative of an electrical connection between the interconnect layer 214 and the electrically conductive dematching layer 200.

Subsequently, at step 218, the substrate layer 202 may be removed. As noted with reference to FIG. 12, techniques, such as, but not limited to, chemical etching, mechanical grinding, or thermal methods may be employed to remove the substrate 202.

As previously described with reference to FIG. 12, electrical ground connections in the transducer assembly illustrated in FIG. 13 may be accomplished via use of a relatively thin foil (not shown), where the relatively thin foil may be laminated to the top of the second matching layer 198, in one embodiment. It may be noted that in certain embodiments both the first matching layer 196 and the second matching layer 198 may be conductive or have micro-vias (not shown) disposed through them to facilitate the ground connections. Alternatively, the first matching layer 196 may be conductive, while one or more micro-vias may be disposed through the second matching layer 198, where the micro-vias may be filled with epoxy (not shown). In certain other embodiments, the ground connections may be accomplished using micro-vias and/or traces that may be disposed along the sides of the individual transducer elements 206 and coupled to pads on the interconnect layer 214.

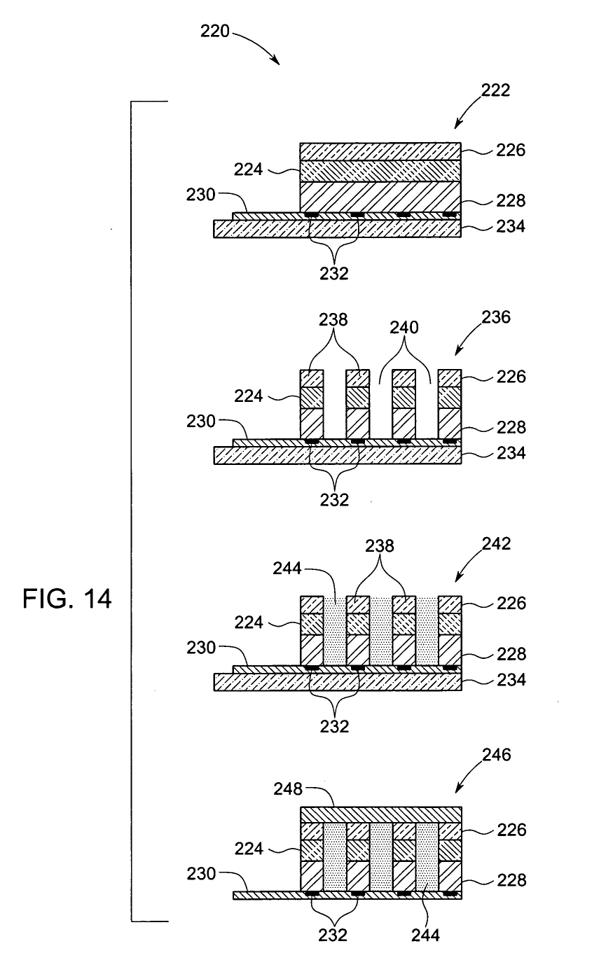

FIG. 14 illustrates a further series of structures in yet another exemplary method 220 for forming a low-profile transducer assembly, in accordance with aspects of the present technique. Step 222 is an initial step in the process 220 where an acoustic stack may be formed by disposing an acoustic layer 224, a first matching layer 226 and a dematching layer 228. The acoustic layer 224 having a top side and a bottom side may be selected. The first matching layer 226 having a top side and a bottom side may then be disposed on the top side of the acoustic layer 224. In addition, a dematching layer 228 having a top side and a bottom side may be disposed on the bottom side of the acoustic layer 224 such that the top side of the dematching layer 228 is in contact with the bottom side of the acoustic layer 224. It may be noted that in the embodiment depicted in FIG. 14, the dematching layer 228 and the first matching layer 226 may be configured to be electrically conductive.

The acoustic stack including the first matching layer 226, the acoustic layer 224 and the dematching layer 228 may then be operatively coupled to an interconnect layer 230 having a top side and a bottom side such that the bottom side of the dematching layer 228 is operatively coupled to the top side of the interconnect layer 230. Subsequently, the stack with the interconnect layer 230 may be bonded to a substrate 234. Alternatively, the substrate 234 and the interconnect layer 230 may be the same piece or layer. Reference numeral 232 is representative of the electrical connection between the interconnect layer 230 and the dematching layer 228.

At step 236, the transducer assembly may be diced to form a plurality of transducer elements 238. Accordingly, one or more saw kerfs 240 may extend through the first matching layer 226, the acoustic layer 224 and the dematching layer 228, and possibly partially into the interconnect layer 230 (not shown).

Further, at step 242, a kerf filler 244 may be disposed in the inter-element space 240 between the plurality of transducer elements 238. The kerf filler 244 may include filled or unfilled silicone or epoxy and may be configured to mechanically strengthen the transducer assembly by filling the inter-element space 240 thereby creating a less fragile and more reliable assembly. The kerf filler 244 may be configured to have low shear stiffness or high shear attenuation, thereby resulting in minimized inter-element cross talk.

Subsequently, at step 246, a second matching layer 248 having a top side and a bottom side may be disposed on the acoustic stack such that the bottom side of the second matching layer 248 is operatively coupled to the top side of the first matching layer 226. In accordance with aspects of the present technique, the second matching layer 248 may include metalization on the bottom side, thereby providing a common ground connection across the array of transducer elements 238. It may also be noted that the second matching layer 248 may optionally be diced into elements that correspond to the elements formed in step 236 where the dicing may only go partially through the second matching layer 248.

Also, at step 246, the substrate layer 234 may be removed. As noted with reference to FIG. 12, techniques, such as, but not limited to, chemical etching, mechanical grinding, or thermal methods may be employed to remove the substrate 234.

It may be noted that the methods of forming the transducer assembly described with reference to FIGS. 12-14 may be employed to form one-dimensional transducer arrays and two-dimensional transducer arrays. Furthermore, the transducer assemblies thus formed may be disposed within the lumen of an invasive probe configured for interventional procedures.

The various low-profile transducer assemblies, invasive probes having the low-profile transducer assemblies for imaging and method of imaging described hereinabove dramatically enhance imaging resolution and sensitivity. The low-profile transducer assembly may be optimized for miniature probes such as catheters for two-dimensional or real-time three-dimensional imaging. The acoustic stack may have a thickness that is reduced by a factor of two or greater relative to conventional acoustic stacks. Additionally, the exemplary transducer assembly described hereinabove does not require an acoustically attenuative backing layer to the rear of the dematching layer as opposed to conventional transducer assemblies that use a low acoustic impedance attenuating backing disposed to the rear of the transducer assembly. Consequently, the transducer assembly may be configured to be relatively thin, thereby allowing the elevational aperture to be as large as possible. In addition, the catheter environment imposes severe space limitations for some applications, particularly for those requiring the passage of additional components beyond the transducer array to the distal tip of the probe. These space limitations are alleviated by the thin, low profile nature of the acoustic stack.

The transducer assembly formed employing the method of forming described hereinabove provides improved image resolution due to the low-profile nature of the transducer assembly, allowing a larger elevational aperture. Additionally, the reduced electrode separation of the relatively thinner acoustic layer results in increased sensitivity. Furthermore, maximizing the elevational aperture of the transducer assembly advantageously results in increased sensitivity due to a larger surface area of the transducer assembly. Also, the low profile of the transducer assembly results in increased area inside the catheter lumen for other components, such as a working port, a fluid passageway, or electrical leads.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

1. A transducer assembly, comprising:
an acoustic layer having a first side and a second side, opposite the first side;
at least one matching layer disposed on the first side of the acoustic layer; and
a dematching layer disposed on the second side of the acoustic layer, wherein the dematching layer has an acoustic impedance greater than an acoustic impedance of the acoustic layer,
wherein the transducer assembly does not include a backing layer that is highly attenuative.

2. The transducer assembly of claim 1, wherein the acoustic layer comprises a plurality of transducer elements.

3. The transducer assembly of claim 2, wherein the acoustic layer comprises lead zirconate titanate, a piezoelectric ceramic, a piezocomposite, a piezoelectric single crystal, or a piezopolymer.

4. The transducer assembly of claim 1, wherein the at least one matching layer has an acoustic impedance less than the acoustic impedance of the acoustic layer.

5. The transducer assembly of claim 1, wherein the dematching layer has a thickness approximately in a range centered around one-fourth of a wavelength of sound in the dematching layer at a frequency of operation of the transducer assembly.

6. The transducer assembly of claim 1, wherein the dematching layer has a thickness approximately in a range centered around one-sixth of a wavelength of sound in the dematching layer at a frequency of operation of the transducer assembly.

7. The transducer assembly of claim 1, further comprising an interconnect layer including at least one conductive element disposed on a substrate having a top side and a bottom side.

8. A transducer assembly configured for use in an invasive probe, consisting of:
an acoustic layer having a first side and a second side, opposite the first side;
at least one matching layer disposed on the first side of the acoustic layer;
a dematching layer disposed on the second side of the acoustic layer, wherein the dematching layer has an acoustic impedance greater than an acoustic impedance of the acoustic layer; and
a interconnect layer including at least one conductive element disposed on a substrate having a top side and a bottom side.

9. An invasive probe configured to image an anatomical region, comprising:
an outer envelope sized and configured to be removably inserted into a patient;
a transducer assembly disposed in the outer envelope, comprising:
an acoustic layer having a first side and a second side, opposite the first side;
at least one matching layer disposed on the first side of the acoustic layer; and
a dematching layer disposed on the second side of the acoustic layer, wherein the dematching layer has an acoustic impedance greater than an acoustic impedance of the acoustic layer,
wherein the transducer assembly does not include a backing layer that is highly attenuative.

10. The invasive probe of claim 9, wherein the invasive probe comprises an imaging catheter, an endoscope, a laparoscope, a surgical probe, a transvaginal probe, a transrectal probe, an intracavity probe, or a probe adapted for interventional procedures.

11. The invasive probe of claim 9, further comprising a working port disposed in the outer envelope and configured to deliver therapy to one or more regions of interest in the patient.

12. The invasive probe of claim 9, further comprising a fluid passageway, one or more electrical lead passageways, or both disposed in the outer envelope.

13. The invasive probe of claim 9, wherein the acoustic layer comprises lead zirconate titanate, a piezoelectric ceramic, a piezocomposite, a piezoelectric single crystal, or a piezopolymer.

14. The invasive probe of claim 9, wherein the dematching layer has a thickness of approximately one-fourth of a wavelength of sound in the dematching layer at a frequency of operation of the transducer.

15. A system, comprising:
an acquisition subsystem configured to acquire image data, wherein the acquisition subsystem comprises an invasive probe configured to image an anatomical region, wherein the invasive probe comprises:
an outer envelope sized and configured to be removably inserted into a patient;
a transducer assembly disposed in the outer envelope, comprising:
an acoustic layer having a first side and a second side opposite the first side;
at least one matching layer disposed on the first side of the acoustic layer;
a dematching layer disposed on the second side of the acoustic layer, wherein the dematching layer has an acoustic impedance greater than an acoustic impedance of the acoustic layer; and
a processing subsystem in operative association with the acquisition subsystem and configured to process the image data acquired via the acquisition subsystem,
wherein the transducer assembly does not include a backing layer that is highly attenuative.

16. The system of claim 15, wherein the processing subsystem comprises an imaging system, wherein the imaging system comprises an ultrasound imaging system adapted for medical or industrial applications.

17. A method for forming a transducer assembly, the method comprising:
forming a stacked structure, the stacked structure comprising:
an acoustic layer having a first side and a second side opposite the first side;
at least one matching layer disposed on the first side of the acoustic layer;
a dematching layer disposed on the second side of the acoustic layer,
wherein the dematching layer has an acoustic impedance greater than an acoustic impedance of the acoustic layer;
bonding the stacked structure to an interconnect layer, or a substrate, or both; and
dicing the stacked structure to form a plurality of transducer elements,
wherein the transducer assembly does not include a backing layer that is highly attenuative.

18. The method of claim 17, further comprising removing the substrate.

19. The method of claim 17, wherein the step of forming the stacked structure comprises:
forming an acoustic layer having a first side and a second side opposite the first side;
disposing at least one matching layer on the first side of the acoustic layer; and
disposing a dematching layer on the second side of the acoustic layer.

20. The method of claim 17, further comprising disposing filler material between the plurality of transducer elements.

21. The method of claim 17, wherein the step of bonding the acoustic structure comprises electrically coupling the transducer assembly to the interconnect layer, or the substrate, or both.
